(19) United States
(12) Patent Application Publication
(10) Pub. No.: US 2018/0344748 A1
(43) Pub. Date: Dec. 6, 2018

(54) METHODS AND COMPOSITIONS FOR PREVENTING OR TREATING CHRONIC INFLAMMATORY DISEASES

(71) Applicant: ENTERIN LABORATORIES, INC., Merion, PA (US)

(72) Inventors: Denise Barbut, New York, NY (US); Ethan Stolzenberg, Glen Rock, NJ (US); Michael Zasloff, Merion, PA (US)

(73) Assignee: ENTERIN LABORATORIES, INC., Merion, PA (US)

(21) Appl. No.: 15/992,963

(22) Filed: May 30, 2018

Related U.S. Application Data
(60) Provisional application No. 62/513,022, filed on May 31, 2017.
(60) Provisional application No. 62/525,667, filed on Jun. 27, 2017.

(51) Int. Cl. A61K 31/575; C07K 16/28; C12N 15/113; A61P 29/00; G01N 33/68

(52) U.S. Cl. CPC A61K 31/575; C07K 16/2845; C12N 15/113; A61K 2039/505; G01N 33/6863; A61K 2300/00; A61P 29/00

(57) ABSTRACT

Described are methods of treating or preventing an inflammatory disease or condition caused by excessive expression, secretion, or concentration of alpha synuclein. The methods comprise administering compositions that reduce the effective concentration of alpha-synuclein present in the surrounding tissues. In addition, the invention encompasses methods of inhibiting alpha-synuclein interaction with CD11b, comprising administering an agent that binds to alpha-synuclein to prevent binding productively to the integrin CD11b, or administering an active agent that interacts with CD11b on an immune cell to prevent alpha-synuclein from directing chemotaxis of that cell. In addition, the invention discloses a method of identifying an individual with a condition amenable to treatment targeting alpha-synuclein CD11b interaction.

### CROSS-REFERENCE TO RELATED APPLICATIONS

The present invention claims priority to U.S. Provisional Application No. 62/513,022, filed on May 31, 2017, and U.S. Provisional Application No. 62/525,667, filed on Jun. 27, 2017, the disclosures of which are specifically incorporated by reference in their entirety.

### BACKGROUND OF THE INVENTION

Alpha-synuclein is a neuronal protein that has excited widespread interest because of its role in the pathogenesis of Parkinson's disease (PD) and the related alpha-synucleinopathies, Lewy Body dementia and Multiple System Atrophy. Of particular interest over the past decade or so has been the scientific debate spurred by the work of Braak, who has shown through his studies of pathological specimens from autopsied individuals at various stages of PD that the accumulation of alpha-synuclein actually begins in the enteric nervous system (ENS) (Del Tredici and Braak, 2016). Alpha-synuclein is also known as SNCA, NACP, PARK1, PARK4, and PD1.

Alpha-synuclein is abundant in the human brain, but smaller amounts are found in the heart, muscles, and other tissues. In the brain, alpha-synuclein is found mainly at the tips of nerve cells (neurons) in specialized structures called presynaptic terminals. Within these structures, alpha-synuclein interacts with phospholipids and proteins. Presynaptic terminals release chemical messengers, called neurotransmitters, from compartments known as synaptic vesicles. The release of neurotransmitters relays signals between neurons and is critical for normal brain function.

Function:

There is no known function for alpha-synuclein. However, studies suggest that it plays a role in maintaining a supply of synaptic vesicles in presynaptic terminals by clustering synaptic vesicles. It may also help regulate the release of dopamine, a type of neurotransmitter that is critical for controlling the start and stop of voluntary and involuntary movements. Alpha-synuclein is specifically upregulated in a discrete population of presynaptic terminals of the brain during a period of acquisition-related synaptic rearrangement. It has been shown that alpha-synuclein significantly interacts with tubulin, and that alpha-synuclein may have activity as a potential microtubule-associated protein, like tau. In particular, alpha-synuclein simultaneously binds to phospholipids of the plasma membrane via its N-terminus domain and to synaptobrevin-2 via its C-terminus domain, with increased importance during synaptic activity. There is growing evidence that alpha-synuclein is involved in the functioning of the neuronal Golgi apparatus and vesicle trafficking. Further, apparently alpha-synuclein is essential for normal development of cognitive functions, as Knock-out mice with targeted inactivation of the expression of alpha-synuclein show impaired spatial learning and working memory.

Structure:

The human alpha-synuclein protein is made of 140 amino acids and is encoded by the SNCA gene. An alpha-synuclein fragment, known as the non-Abeta component (NAC) of AD amyloid, originally found in an amyloid-enriched fraction, was shown to be a fragment of its precursor protein, NACP. It was later determined that NACP was the human homologue of Torpedo synuclein. Therefore, NACP is now referred to as human alpha-synuclein. Ueda et al. (1993). Alpha-synuclein in solution is considered to be an intrinsically disordered protein, i.e., it lacks a single stable 3D structure. van Rooijen et al. (2009). As of 2014, an increasing number of reports suggest, however, the presence of partial structures or mostly structured oligomeric states in the solution structure of alpha-synuclein, even in the absence of lipids. At least three isoforms of synuclein are produced through alternative splicing. The majority form of the protein is the full-length protein of 140 amino acids. Other isoforms are (i) alpha-synuclein-126, which lacks residues 41-54 due to loss of exon 3; and (ii) alpha-synuclein-112, which lacks residue 103-130 due to loss of exon 5. Alpha-synuclein's primary structure is usually divided in three distinct domains: (1) residues 1-60, which is an amphipathic N-terminal region dominated by four 11-residue repeats including the consensus sequence KTKEGV. This sequence has a structural alpha helix propensity similar to apolipoproteins-binding domains; (2) residues 61-95, which is a central hydrophobic region which includes the non-amyloid-β component (NAC) region, involved in protein aggregation; and (3) residues 96-140, which is a highly acidic and proline-rich region which has no distinct structural propensity.

Significance:

Classically considered an unstructured soluble protein, unmutated α-synuclein forms a stably folded tetramer that resists aggregation. This observation is still a matter of debate in the field due to conflicting reports. Nevertheless, alpha-synuclein aggregates to form insoluble fibrils in pathological conditions characterized by Lewy bodies, such as PD, dementia with Lewy bodies, and multiple system atrophy. These disorders are known as synucleinopathies. Alpha-synuclein is the primary structural component of Lewy body fibrils. Occasionally, Lewy bodies contain tau protein; however, alpha-synuclein and tau constitute two distinctive subsets of filaments in the same inclusion bodies. Alpha-synuclein pathology is also found in both sporadic and familial cases with AD.

The aggregation mechanism of alpha-synuclein is uncertain. There is evidence of a structured intermediate rich in beta structure that can be the precursor of aggregation and, ultimately, Lewy bodies. Among the strategies for treating synucleinopathies are compounds that inhibit aggregation of alpha-synuclein. It has been shown that the small molecule cuminaldehyde inhibits fibrillation of alpha-synuclein. In rare cases of familial forms of PD, there is a mutation in the gene coding for alpha-synuclein. Five alpha-synuclein point mutations have been identified thus far: A53T, A30P, E46K, H50Q, and G51D.

Because it physically associates with vesicular structures, such as synaptic vesicles, alpha-synuclein is described as a protein that is somehow involved in neurotransmitter release (Burre, 2015). Recently, human alpha-synuclein was shown to chemo-attract rodent microglia, suggesting that alpha-synuclein could directly promote neuro-inflammatory damage within the CNS (Wang et al., 2015). In addition, alpha-synuclein has also recently been shown to be protective in mice infected with by neurotropic RNA viruses, such as West Nile Virus (WNV) and Venezuelan Equine Encephalitis (Beatman et al., 2015).

Inflammatory Diseases and Conditions:

Inflammatory diseases represent a diverse spectrum of conditions sharing the common feature of accumulation of inflammatory cells in the soft tissues of the body. The precise anatomic localization of inflammation varies depending on the disease. For instance, in rheumatoid arthritis, inflammation occurs within the tissues that surround the joints, called the synovial membrane. In psoriasis, inflammation occurs within the dermis of the skin. In Crohn's disease, inflammation is seen within the wall of the gastrointestinal tract, and in polymyositis, inflammation is seen in the muscles.

In general, inflammatory diseases are treated by either reducing the population of inflammatory cells within the body or by suppressing the production of the damaging proteins secreted by the inflammatory cells that have accumulated within a tissue. For instance, corticosteroids can both dramatically reduce the production of lymphocytes in the body as well as inhibit the production of cytokines by existing cells. Antibodies such as basiliximab (Simulect®) massively deplete immune cells and effect extensive immunosuppression. Drugs such as infliximab (Remicade®) target TNF-alpha, a protein that both attracts and activates inflammatory cells within soft tissues. More recently, new drugs target other pro-inflammatory proteins or their receptors, such as IL-23 and its receptor.

Aminosterols:

An exemplary aminosterol is squalamine, which is a sulfated aminosterol originally detected in Squalus acanthias (spiny dogfish shark) due to its broad-spectrum antimicrobial (antibiotic) activity. Aminosterols such as squalamine are known to have antibiotic activity, as well as antiangiogenesis activity, appetite suppression, or inhibition of sodium proton exchanger proteins. Shinnar et al. (2007). Further analyses of larger quantities of dogfish liver extracts revealed squalamine to be the most abundant member of a larger aminosterol family comprising at least 12 related compounds. Rao et al., (2000). A known squalamine derivative is Aminosterol 1436, also known as MSI-1436 and trodusquemine. Although structurally similar to squalamine (it carries a spermine rather than a spermidine) and also quite potent as an anti-infective, Aminosterol 1436 exhibits a profoundly different pharmacology in vertebrates, causing weight loss and adipose tissue mobilization. Zasloff et al. (2001). Prior to the present invention, it was not known that aminosterols such as squalamine and aminosterol 1436 have an effect on alpha-synuclein expression and/or secretion.

Squalamine was initially discovered on the basis of its anti-bacterial activity. It has proven to be a broad spectrum antimicrobial compound that exhibits potent activity in vitro and in vivo against gram negative and gram positive bacteria, fungi, protozoa, and many viruses. Subsequent studies demonstrated that squalamine exhibits systemic anti-angiogenic activity against rapidly proliferating blood vessels that arise in pathological settings. As a consequence it is being evaluated in several human clinical trials for cancer, macular degeneration, diabetic retinopathy, and fibrodysplasia ossificans progressiva.

There is a need in the art for methods and compositions that target treatment and prevention of inflammatory conditions and related diseases caused by excessive expression of alpha synuclein. The present invention satisfies these needs.

### SUMMARY OF THE INVENTION

The present invention relates to novel methods of treating and/or preventing inflammatory diseases and conditions caused by excessive expression of neuronal alpha-synuclein. The methods are based on the discovery that alpha-synuclein is a potent pro-inflammatory hormone. The invention comprises methods that target alpha-synuclein, by reducing the concentration of alpha-synuclein within tissues, and/or by blocking the interaction of alpha-synuclein with receptors on inflammatory cells. In addition, the invention provides a method of identifying individuals, or a specific patient population, that could benefit from the treatment and/or prevention of diseases caused by excessive expression of neuronal alpha-synuclein.

Thus, the present invention is directed to the discovery that inflammation can be blocked by either of two strategies. First, inflammation can be blocked by reducing the tissue concentration of alpha-synuclein by decreasing or stopping production of alpha-synuclein. Alternatively, inflammation can be blocked by interrupting the signaling between alpha-synuclein and inflammatory cells that express CD11b. The subject of the methods of the invention can be any mammal, including a human.

In one embodiment, the invention is directed to methods of treating or preventing an inflammatory disease or condition caused by excessive expression of alpha synuclein in a subject. The method comprises administering to the subject a composition that results in reducing the concentration of alpha-synuclein in tissue or at a site of inflammation. The composition can be administered via any pharmaceutically acceptable method. In one embodiment, the composition that reduces the concentration of alpha synuclein comprises an effective amount of an aminosterol, miR-34b, or miR-34c. The compositions of the invention can further comprises at least one pharmaceutically acceptable carrier.

In another embodiment, the composition that results in reducing the tissue concentration of alpha-synuclein comprises an aminosterol, and can further comprise a pharmaceutical excipient. In yet another embodiment of the invention, the aminosterol is squalamine or a derivative thereof, such as aminosterol 1436. Examples of other compounds known to inhibit alpha-synuclein expression include miR-34b and miR-34c. Kabaria et al. (2015).

In one embodiment, the tissue is from a site of inflammation. Exemplary tissues in which the concentration of alpha-synuclein can be measured include those commonly involved in human inflammatory conditions, such as the GI tract, skin, lungs, liver, kidney, heart, and joint synovial membranes. In one embodiment, the tissue evaluated and measured for alpha-synuclein concentration is from a site of inflammation. The reduction in alpha-synuclein concentration can be measured as compared to a control or as compared to a tissue concentration measured in the same tissue type from the same subject prior to treatment.

In one embodiment of the invention, wherein the decrease in alpha-synuclein concentration in is measured qualitatively, quantitatively, or semi-quantitatively by one or more methods selected from the group consisting of: (a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control; (b) measuring the intensity of inflammation over time; (c) measuring the amount of inflammatory markers over time; (d) measuring the amount of inflammatory markers in blood, plasma, or tissue over time, either qualitatively or quantitatively; (e) measuring the amount of one or more inflammatory marker cytokines in blood, plasma, or tissue over time, either qualitatively or quantitatively; (f) measuring the amount of one or more plasma markers of inflammation such as TNF, IL-8, or CRP in blood, plasma, or tissue over time, either qualitatively or quantitatively; or (g) measuring the amount of inflammatory cells in blood, plasma, or tissue over time, either qualitatively or quantitatively.

For example, the decrease in alpha-synuclein concentration in tissue can be measured by first determining the concentration of alpha-synuclein in a tissue sample prior to treatment, followed by determining the alpha-synuclein concentration in the same tissue type following treatment. Alternatively, the alpha-synuclein concentration after treatment in a tissue can be compared to the concentration of alpha-synuclein in the same tissue type from a control, e.g., a healthy subject.

The methods of determining alpha-synuclein concentration in a tissue can include quantitative and semi-quantitative analytical techniques, such as ELISA, immunohistochemistry, protein electrophoresis, chromatography, Western blotting, PCR, and other quantitative measures of either protein or nucleic acid concentrations. Any pharmaceutically acceptable method of determining alpha-synuclein concentration in a tissue can be utilized in the methods of the invention.

The methods of the invention can result in a decrease in expression, production, or secretion of alpha synuclein, or a decrease in the concentration of alpha-synuclein in a tissue or at a site of inflammation, or a combination thereof. The methods of the invention can also result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. The decrease can be of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The aminosterol used in the methods of the invention can be a natural aminosterol isolated from the liver of Squalus acanthias, e.g., squalamine or a salt thereof, or a synthetic version thereof. In another embodiment, the aminosterol used in the methods of the invention can be a squalamine isomer. The aminosterol can be Aminosterol 1436 or a salt or derivative thereof. In yet another embodiment, the aminosterol used in the methods of the invention can comprise a sterol nucleus and a polyamine, attached at any position on the sterol, such that the molecule exhibits a net charge of at least +1, the charge being contributed by the polyamine. The aminosterol used in the methods of the invention can also comprise a bile acid nucleus and a polyamine, attached at any position on the bile acid, such that the molecule exhibits a net charge of at least +1, the charge being contributed by the polyamine. Further, the aminosterol used in the methods of the invention can be modified to include one or more of the following: (a) substitutions of the sulfate by a sulfonate, phosphate, carboxylate, or other anionic moiety chosen to circumvent metabolic removal of the sulfate moiety and oxidation of the cholesterol side chain; (b) replacement of a hydroxyl group by a non-metabolizable polar substituent, such as a fluorine atom, to prevent its metabolic oxidation or conjugation; or (c) substitution of one or more ring hydrogen atoms to prevent oxidative or reductive metabolism of the steroid ring system. Finally, the aminosterol used in the methods of the invention can be a derivative of squalamine or natural aminosterol modified through medical chemistry to improve bio-distribution, ease of administration, metabolic stability, or any combination thereof.

In the methods of the invention utilizing an aminosterol, any pharmaceutically acceptable dose of an aminosterol can be used. For example, the effective daily dose of the aminosterol can be about 0.1 to about 20 mg/kg body weight, or any body weight in-between these two values, e.g., about 0.5, about 5, about 10, about 15 or about 20 mg/kg body weight. Other exemplary dosages of an aminosterol are described herein and include, for example, (a) about 0.1 to about 150 mg/kg body weight; (b) about 10 to about 100 mg/subject; (c) about 10 mg to about 400 mg/subject; (d) about 25 to about 1000 mg/subject; (e) about 25 to about 500 mg/subject; (f) about 50 to about 350 mg/subject; or (g) about 1 to about 110 mg/m².

In another embodiment, encompassed are methods of treating or preventing an inflammatory disease or condition caused by excessive expression of neuronal alpha synuclein, comprising administering a composition comprising an active agent that forms a physical complex with alpha-synuclein, thereby preventing the interaction of alpha-synuclein with CD11b. For example, the administered composition can comprise a molecule that complexes with either monomeric or polymeric alpha-synuclein to inhibit binding of alpha-synuclein to CD11b. The inhibition of binding to CD11b functions to curb the stimulus that draws inflammatory cells into the tissues. Molecules such as antibodies or compounds that exhibit a high affinity for alpha-synuclein are recognized as appropriate molecules for use in these methods of the invention. An exemplary active agent or compound is an antibody that specifically binds to alpha-synuclein, forming a physical complex. Any pharmaceutically acceptable dose of an appropriate molecule can be used in the methods of the invention.

In yet another embodiment, encompassed are methods of treating or preventing an inflammatory disease or condition caused by excessive expression of neuronal alpha synuclein. The method comprises administering a composition comprising an active agent that binds to CD11b, thereby inhibiting the binding of alpha-synuclein to CD11b. Such a compound is designed to selectively block the binding sites on CD11b that interact with alpha-synuclein but not necessarily interfere with the function of CD11b with respect to its response to other ligands to which it normally responds. An exemplary active agent or compound is an antibody that specifically binds to CD11b, forming a physical complex. Any pharmaceutically acceptable dose of an appropriate molecule can be used in the methods of the invention.

The methods of treating or preventing an inflammatory disease or condition caused by excessive expression of neuronal alpha synuclein, comprising blocking alpha-synuclein CD11b signaling by (i) administering a composition comprising an active agent that forms a physical complex with alpha-synuclein, or (ii) administering a composition comprising an active agent that binds to CD11b, can utilize a different method for determining success (e.g., rather than measuring alpha-synuclein concentration). Specifically, the effectiveness of blocking alpha-synuclein CD11b signaling can be measured indirectly either qualitatively or quantitatively by measuring a decrease in inflammation by, for example, (1) measuring the intensity of inflammation over time, with a decrease in inflammation intensity correlating with successful treatment (a qualitative determination); (2) monitoring blood levels of inflammatory markers over time, with a decrease in inflammatory markers correlating with successful treatment (a qualitative or quantitative determination) (exemplary markers can include cytokines or plasma markers of inflammation such as TNF, IL-8, or CRP); (3) measurement of inflammatory markers in tissue via biopsy over time, with a decrease in inflammatory markers in tissue correlating with successful treatment (a qualitative or quantitative determination) (exemplary markers can include cytokines or plasma markers of inflammation such as TNF, IL-8, or CRP); or (4) measurement or monitoring of the number of inflammatory cells in tissue, with a decrease in the number of inflammatory cells in tissue correlating with successful treatment (a qualitative or quantitative determination). Thus, the decrease in tissue inflammation can measured by: (a) measuring the intensity of inflammation over time; (b) measuring the amount of inflammatory markers over time; (c) measuring the amount of inflammatory markers in blood, plasma, or tissue over time, either qualitatively or quantitatively; (d) measuring the amount of one or more inflammatory marker cytokines in blood, plasma, or tissue over time, either qualitatively or quantitatively; (e) measuring the amount of one or more plasma markers of inflammation such as TNF, IL-8, or CRP in blood, plasma, or tissue over time, either qualitatively or quantitatively; or (f) measuring the amount of inflammatory cells in blood, plasma, or tissue over time, either qualitatively or quantitatively.

The compositions of the invention can be administered via any pharmaceutically acceptable method. For example, the pharmaceutical composition in the methods of the invention can be administered intravenously, intradermally, subcutaneously, orally, rectally, sublingually, intrathecally, intranasally, or by inhalation. Pharmaceutical compositions appropriate for each of the specific routes are utilized.

The methods of the invention can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The inflammatory disease or condition caused by excessive expression of neuronal alpha synuclein can be a neurodegenerative disorder (NDD), such as an alpha-synucleinopathy. Exemplary alpha-synucleinopathies include, but are not limited to, Parkinson's disease, Lewy body dementia, multiple system atrophy, amytrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal alpha synuclein can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases.

Further, in the methods of the invention utilizing an aminosterol, the aminosterol can be administered in combination with at least one additional active agent to achieve either an additive or synergistic effect. The additional active agent can be administered via a method selected from the group consisting of concomitantly; as an admixture; separately and simultaneously or concurrently; and separately and sequentially. The additional active agent can be an agent known to be useful in treating the condition or disease targeted for treatment by the method.

In addition, it follows from the present invention that an individual with an inflammatory condition appropriate for treatment or prophylaxis with the methods targeting alpha-synuclein described herein can be identified by determination of the tissue concentrations of alpha synuclein at sites of inflammation, with high levels of alpha-synuclein, as compared to a control or healthy subject, correlating with patients appropriate for treatment with a method of the invention.

In another method of the invention, encompassed is a method of identifying a subject with a condition amenable to treatment targeting alpha-synuclein CD11b interaction. The method comprises identifying a subject having an elevated concentration of alpha-synuclein present in a tissue, using either qualitative, quantitative, or semi-quantitative methods. For example, the method can comprise: (a) obtaining a tissue sample from a site of inflammation from the subject; and (b) qualitatively, quantitatively or semi-quantitatively determining the concentration of alpha synuclein within the tissue sample; wherein an elevated concentration of alpha-synuclein present in the tissue, as compared to a control or healthy subject, indicates that the subject is amenable to treatment targeting alpha-synuclein CD11b interaction. Other suitable methods of identifying subjects having an elevated concentration of alpha-synuclein present in a tissue are described herein and can also be used in the methods of the invention. For example, a subject amenable to treatment using methods of the invention can be identified by (a) measuring the intensity of inflammation over time; (b) measuring the amount of inflammatory markers over time; (c) measuring the amount of inflammatory markers in blood, plasma, or tissue over time, either qualitatively or quantitatively; (d) measuring the amount of one or more inflammatory marker cytokines in blood, plasma, or tissue over time, either qualitatively or quantitatively; (e) measuring the amount of one or more plasma markers of inflammation such as TNF, IL-8, or CRP in blood, plasma, or tissue over time, either qualitatively or quantitatively; or (f) measuring the amount of inflammatory cells in blood, plasma, or tissue over time, either qualitatively or quantitatively.

Both the foregoing summary of the invention and the following brief description of the drawings and the detailed description of the invention are exemplary and explanatory and are intended to provide further details of the invention as claimed. Other objects, advantages, and novel features will be readily apparent to those skilled in the art from the following detailed description of the invention.

### DESCRIPTION OF THE FIGURES

FIGS. 1A-I: Shown are representative biopsies from the pediatric population immunostained for α-synuclein, PGP 9.5, and CD68. The panels labelled αS (FIG. 1A) and PGP 9.5 (FIG. 1D) on the top and bottom left were serial sections (20×). Inset 1 (FIG. 1E) highlights an area of reduced α-synuclein staining adjacent to an area (inset 3) (FIG. 1C) of robust expression (inset 2) (FIG. 1B). Neurites are evident in both areas (insets 3 and 4) (FIGS. 1C and 1F), highlighting the focal nature of α-synuclein deposition in the duodenum. The panels labelled αS (FIG. 1G) and CD68 (FIG. 1H) are serial sections immunostained for α-synuclein and CD68 (20×), highlighting the presence of chronic inflammatory cells in regions of the specimen in which α-synuclein expression is seen. FIG. 1I shows a graph of the intensity of acute and chronic inflammation correlates with intensity of α-synuclein staining. α-Synuclein intensities of 1-2, and 3-4 scored for each of the sections were grouped into low (black bars) and high (grey bars) groups, respectively. The degree of infiltration of neutrophils (Acute) and or mononuclear cells (Chronic) in the corresponding sections is plotted relative to intensity of α-synuclein expression. Statistical significance was determined by Student's t-test.

FIGS. 2A-F: Shown are representative biopsies from intestinal transplant recipients with documented Norovirus infection stained for α-synuclein (20×). FIGS. 2A, 2B, and 2C show results from Case 16, native duodenum. FIG. 2A shows minimal relative expression of α-synuclein in the specimen from case 16, taken 1 month prior to the Norovirus infection (Case 16, Pre). FIG. 2B shows robust expression of α-synuclein was seen during the viral expression (Case 16, During). No clinical evidence of any infection was noted during the 1 month period prior to the Norovirus infection. FIG. 2C shows an inset of an enlargement of a section of FIG. 2B. FIGS. 2D, 2E, and 2F show results from Case 3 (Allograft jejunum). FIG. 2D shows areas of high focal expression of α-synuclein during the Norovirus infection (Case 3, During). FIG. 2E shows approximately the same region of the allograft, biopsied 4 months later, the patient still PCR+ for Norovirus (Case 3, Post). Finally, FIG. 2F shows an inset of an enlargement of a section of FIG. 2E.

FIGS. 3A-F: α-Synuclein is a chemoattractant dependent on CD11b. Assays were conducted as described in the examples below. The average of 3 independent assays for each sample is presented. The concentration of the α-synuclein aggregate is in terms of the monomer. A positive control, FMLP, is noted in black. NAc 1-21, the N-acetylated peptide corresponding to the first 21 amino acids of α-synuclein; 1-25, a peptide corresponding to the first 25 amino acids. FIG. 3A=Human Neutrophils; FIG. 3B=Human monocytes; FIGS. 3C and 3D=Representative photos of the cellular response. FIG. 3E=Neutrophils from CD11b −/− or wild type mice (0.5×106/ml), assayed as described in the examples. FIG. 3F=Human neutrophils incubated in the presence of anti-CD11b antibody or control (50 μg/ml each). CM, control media (negative control)

FIG. 4: Human monocyte derived dendritic cells were incubated at 5×10⁵/ml in the presence of recombinant monomeric or aggregated alpha-synuclein, and the NAc 1-21 amino terminal peptide of alpha-synuclein (each at a final concentration of 10 μM) for 48 h before they were immunostained and analyzed by flow cytometry for the expression of the indicated surface molecules. E. coli lipopolysaccharide (LPS) (100 ng/ml) was included as a positive control. Shown are the results of one experiment representative of three. The ordinate of each flow cytometric analysis corresponds to fluorescent intensity/cell, the abscissa to cell number in log scale.

### DETAILED DESCRIPTION OF THE INVENTION

### I. Overview of the Invention

Current treatments of inflammatory diseases and conditions, including chronic inflammatory diseases and conditions, rarely target the primary source of the inflammatory stimulus, namely the primary stimulus that initiates the inflammatory cascade. The present invention is therefore a significant and dramatic improvement over current conventional anti-inflammatory treatments, as the present invention targets the primary stimulus that initiates the inflammatory cascade, rather than just targeting symptoms of the inflammatory cascade. In particular, the present invention discloses that alpha-synuclein, synthesized within and secreted from nerves, represents a primary initiating stimulus of inflammation. Thus, the present invention teaches that by administering a drug that targets alpha-synuclein, inflammation can be minimized, greatly reduced, or suppressed, either by suppressing expression or production of alpha-synuclein or by blocking interaction of alpha-synuclein with the receptor CD11b.

Alpha-synuclein exhibits potent chemo-attractive activity for monocytes and neutrophils. All cells that alpha-synuclein can attract must express CD11b, a protein that permits the cells to attach to the tissue and migrate towards the source of alpha-synuclein. In addition to monocytes and neutrophils, CD11b (positive) cells include dendritic cells, eosinophils, macrophages, NK cells, and certain subsets of T lymphocytes. In other words, alpha-synuclein has the capacity to galvanize a complex population of inflammatory cells at a site of release from a nerve.

CD11b, also known as Mac-1 α or integrin αM chain, is an integrin family member which pairs with CD18 to form the CR3 heterodimer. CD11b is expressed on the surface of many leukocytes including monocytes, neutrophils, NK cells, granulocytes and macrophages, as well as on 8% of spleen cells and 44% of bone marrow cells. Functionally, CD11b regulates leukocyte adhesion and migration to mediate the inflammatory response. CD11b antibody studies have shown the protein to be directly involved in cellular adhesion, although migration can only take place in the presence of the CD18 subunit. Research using CD11b antibodies has identified CD11b as a receptor for fibrinogen gamma chain, factor X and ICAM1, with possible roles in cell-mediated cytotoxicity, chemotaxis and phagocytosis.

In particular, the present invention is based on the discovery detailed herein that α-synuclein is a potent chemoattractant for CD11b positive cells, such as monocytes and neutrophils. In addition, α-synuclein can direct the maturation of dendritic cells, thereby permitting them to effectively participate in an orchestrated immune scenario. Thus, anti-inflammatory treatment targeting alpha-synuclein can involve (i) reduction in alpha-synuclein synthesis, alpha-synuclein secretion, or both; (ii) inactivation of alpha-synuclein through the action of an antibody or a molecule with a high binding affinity for alpha-synuclein; or (iii) an agent that can effectively inhibit the binding of alpha-synuclein to CD11b.

Accordingly, the present invention is directed to the discovery that inflammation can be blocked by either of two strategies. First, inflammation can be blocked by reducing the tissue concentration of alpha-synuclein by administering a composition that results in decreasing or stopping production of alpha-synuclein. Alternatively, inflammation can be blocked by interrupting the signaling between alpha-synuclein and inflammatory cells that express CD11b. The subject of the methods of the invention can be any mammal, including a human.

The reduction in alpha-synuclein synthesis and/or secretion, or inactivation of alpha-synuclein, can be measured by detecting a decrease in the concentration of alpha-synuclein in tissues, either qualitatively or quantitatively. For example, the concentration of alpha-synuclein can be measured in tissue before treatment, and then in the same tissue from the same subject following treatment. Alternatively, the concentration of alpha-synuclein can be measured in a patient before and/or after treatment and compared to a control or healthy subject, with a decreased amount of alpha-synuclein following treatment with a method of the invention correlating with successful treatment. A decrease in alpha-synuclein concentration as described herein correlates with successful treatment or prevention of an inflammatory disease or condition. Exemplary tissues that can be evaluated in include tissues from sites of inflammation. Such tissues would include those commonly involved in human inflammatory conditions, such as the GI tract, skin, lungs, liver, kidney, heart, and joint synovial membranes.

Alternatively, the methods of treating or preventing an inflammatory disease or condition caused by excessive expression of neuronal alpha synuclein, comprising blocking alpha-synuclein CD11b signaling by (i) administering a composition comprising an active agent that forms a physical complex with alpha-synuclein, or (ii) administering a composition comprising an active agent that binds to CD11b, can utilize a different method for determining success (e.g., rather than measuring alpha-synuclein concentration in tissue). Specifically, the effectiveness of blocking alpha-synuclein CD11b signaling can be measured indirectly either qualitatively or quantitatively by, for example, (1) measuring the intensity of inflammation over time, with a decrease in inflammation intensity correlating with successful treatment (a qualitative determination); (2) monitoring blood levels of inflammatory markers over time, with a decrease in inflammatory markers correlating with successful treatment (a qualitative or quantitative determination); (3) measurement of inflammatory markers in tissue via biopsy over time, with a decrease in inflammatory markers in tissue correlating with successful treatment (a qualitative or quantitative determination); or (4) measurement or monitoring of the number of inflammatory cells in tissue, with a decrease in the number of inflammatory cells in tissue correlating with successful treatment (a qualitative or quantitative determination).

The methods of the invention can result in a decrease in alpha-synuclein tissue concentration, intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Utilizing the vagus as an escalator, alpha-synuclein produced in the ENS traffics from the gut to the brain, spreading to centers within the central nervous system (CNS) that ultimately are destroyed. Why it would accumulate in the ENS is unknown. Based on the inventors' discovery that alpha-synuclein accumulates in the nerve fibers of the gastrointestinal tract in proportion to the degree of acute and chronic inflammation occurring within the area of tissue innervated by the alpha-synuclein-rich nerves, it was hypothesized that alpha-synuclein might exhibit pro-inflammatory activity. This indeed was the case (see Examples below).

The intestinal mucosa of vertebrates is a multifunctional organ that enables digestion and absorption of nutrients while simultaneously acting as a barrier to pathogens. When microorganisms breach the epithelium and enter the lamina propria, an innate immune response ensues with the release of cytokines, chemokines and complement factors and the activation and influx of neutrophils, macrophages and dendritic cells. The enteric nervous system is increasingly recognized to modulate this dynamic milieu by releasing neurotransmitters and neuropeptides that can locally alter the immune response.

Inhibition of Binding Alpha-Synuclein to CD11b:

In one embodiment, the invention is directed to methods of preventing or treating an inflammatory disease or condition caused by excessive expression of neuronal alpha-synuclein. The method comprises administering a molecule that complexes with either monomeric or polymeric alpha-synuclein to inhibit interacting with or binding of alpha-synuclein to CD11b. The inhibition of binding to or interacting with CD11b functions to curb the stimulus that draws inflammatory cells into the tissues. Molecules such as antibodies or compounds that exhibit a high affinity for alpha-synuclein are recognized as appropriate molecules for use in these methods of the invention. For example, antibodies against alpha-synuclein are known in the art. Fujiwara et al. (2002). As detailed above, the inhibition of binding to or interacting with CD11b can be measured by monitoring the magnitude of cellular infiltration of inflammatory cells in the previously inflamed tissues. These tissues would be sampled from those commonly involved in human inflammatory conditions, such as the GI tract, skin, lungs, liver, kidney, heart, and joint synovial membranes. Alternatively, other markers of inflammation can be measured, as described above and herein. Any number of routine measures of inflammation can be used, including pro-inflammatory cytokines or the cellular density of inflammatory cells. In addition, the efficacy of the response can be evaluated by measurement of routine plasma markers of inflammation, such as TNF, IL-6, or CRP.

In another embodiment, the invention is directed to a method of preventing or treating an inflammatory disease caused by excessive expression of alpha synuclein, comprising administering a molecule that binds to CD11b to inhibit alpha-synuclein from interacting with or binding to CD11b. Such a compound is designed to selectively block the binding sites on CD11b that interact with alpha-synuclein but not necessarily interfere with the function of CD11b with respect to its response to other ligands to which it normally responds. As detailed above, the inhibition of binding to or interacting with CD11b can be measured by monitoring the magnitude of cellular infiltration of inflammatory cells in the previously inflamed tissues. These tissues would be sampled from those commonly involved in human inflammatory condition, such as the GI tract, skin, lungs, liver, kidney, heart, and joint synovial membranes. Any number of routine measures of inflammation can be used, including pro-inflammatory cytokines or the cellular density of inflammatory cells. In addition, the efficacy of the response can be evaluated by measurement of routine plasma markers of inflammation, such as TNF, IL-6, or CRP.

Inhibition of Production or Secretion of Alpha-Synuclein:

The invention also encompasses a method of preventing or treating an inflammatory disease or condition caused by excessive expression of neuronal alpha synuclein, comprising administering a molecule that inhibits the production and/or the secretion of alpha-synuclein from a neuron. In one embodiment of this invention, the molecule is the aminosterol squalamine, the derivative aminosterol 1436, a salt thereof, or other aminosterol derivatives as defined herein. Examples of other compounds known to inhibit alpha-synuclein expression include miR-34b and miR-34c. Kabaria et al. (2015). As detailed above, the inhibition of production and/or secretion of alpha-synuclein can be measured by detecting a decrease in the concentration of alpha-synuclein in tissues.

The methods of the invention can result in a decrease in expression of alpha synuclein, or decrease in concentration of alpha-synuclein in tissue, or a decrease in another measure of inflammation, such as pro-inflammatory cytokines, the cellular density of inflammatory cells or routine plasma markers of inflammation, such as TNF, IL-6, or CRP. The decrease can be as compared to a control or as compared to a tissue or biological sample from the same subject (and same tissue/biological sample type) prior to treatment. The measurement of the decrease can be qualitative or quantitative. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

As a non-limiting example, the concentration of alpha-synuclein can be measured in tissue—e.g., at a site of inflammation—prior to treatment. Following treatment, the concentration of alpha-synuclein can be measured in the same tissue or tissue location. The “control” can be a measurement of alpha synuclein prior to application of the methods of the invention, and the measurement in reduction of alpha synuclein can be done at any time following treatment.

The time frame over or during which the decrease in expression of alpha synuclein, or decrease in concentration of alpha-synuclein in tissue, or a decrease in another measure of inflammation, such as pro-inflammatory cytokines, the cellular density of inflammatory cells or routine plasma markers of inflammation, such as TNF, IL-6, or CRP, is measured can be any suitable time frame. For example, the time frame can be about 1 week, about 2 weeks, about 3 weeks, about 1 month, about 1.5 months, about 2 months, about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months, or about 12 months following initial administration of a compound or composition in a method of the invention.

Patient Populations:

In some embodiments of the invention, patient populations particularly susceptible to excessive production or secretion of alpha-synuclein, resulting in excessive amounts of alpha-synuclein, can benefit from the methods of the invention and are targeted for therapy. For example, a patient population having a mutated form of alpha-synuclein resulting in increased amounts of alpha-synuclein can be treated using the methods of the invention. Another example of a patient population susceptible for high levels of alpha-synuclein are patients having chronic inflammatory conditions or diseases.

Another example of a patient population that may benefit from the methods of the invention include immunosuppressed patients, including patients having immunosuppression associated with a disease as well as patients having immunosuppression associated with drug therapy (e.g., transplant patients).

In rare cases of familial forms of Parkinson's disease, there is a mutation in the gene coding for alpha-synuclein. Five point mutations in alpha-synuclein have been identified thus far: A53T,[56] A30P,[57] E46K,[58] H50Q,[59] and G51D. It has been reported that some mutations influence the initiation and amplification steps of the alpha-synuclein aggregation process. Patient populations having one or more of these alpha-synuclein point mutations can be treated using the methods of the invention.

Overview of Invention:

α-Synuclein binds to lipid bilayers and associates with synaptic vesicles in the nerve terminal. In the CNS, α-synuclein can activate the innate immune system and induce chemotaxis. α-Synuclein stimulates microglial activation the extent of which correlates with its extracellular deposition. In PD brains, activated microglia surround degenerating neurons in the SN. The mechanism that accounts for this observation was only recently explained when it was shown that α-synuclein aggregates exhibit chemoattractant activity towards murine brain microglia dependent on the integrin CD11b subunit. Wang et al. (2015).

Given the growing body of evidence linking aggregated α-synuclein to immune cell activation and chemotaxis in the brain, it was hypothesized that α-synuclein might play a role in the innate immune defenses of the gastrointestinal tract. Data described herein demonstrates the accuracy of this hypothesis.

Data Summary:

As described in the examples below, in gastrointestinal biopsies from children with upper gastrointestinal symptoms and from intestinal transplant recipients, enteric α-synuclein was evident in most cases where an infection was identified. In 4 of the transplant patients, α-synuclein was absent prior to viral infection and appeared during viral infection. In vitro monomeric and oligomeric α-synuclein had potent CD11b-dependent chemoattractant activity, causing migration of human neutrophils and monocytes. α-Synuclein also stimulated the maturation of human monocyte derived dendritic cells, in vitro. These findings disclose a previously unknown interaction between the nervous and immune systems and suggest that a primary function of α-synuclein is to mobilize the innate immune system during infection.

To determine if α-synuclein expression in the enteric nervous system (ENS) is associated with infection and/or inflammation, biopsies taken over a 9-year period from children were evaluated for clinically significant upper GI distress warranting endoscopy. A pediatric population was selected because it would avoid the bias of an adult population with “pre-clinical Parkinson's disease (PD).” Most of the 42 pediatric cases exhibited pathological evidence of acute and/or chronic mucosal inflammation, as determined by the presence of neutrophil or mononuclear cell infiltrates, respectively. An infectious organism could be implicated as the cause of an inflammatory response in 23/42 (55%) and included H. pylori in 20/23 (87%) and Candida in 2/23 (9%). The biopsies were immunostained for α-synuclein.

α-Synuclein was expressed in 19/23 (83%) of duodenal biopsies with a confirmed bacterial or fungal infection. By contrast, in a recent report, α-synuclein staining in the ENS of the gastric mucosa of subjects over the age of 70 was noted in about 60% of individuals with PD, but in fewer than 10% of an aged matched control population. Sanchez-Ferro et al. (2015). In all tissue specimens examined, neuronal processes that expressed α-synuclein could be seen adjacent to neighboring neuronal processes that did not. Generally, the distribution of macrophages within the lamina propria overlapped with the areas in which α-synuclein was expressed and the inflammatory cell density tended to be proportional to the intensity and extent of nearby α-synuclein staining, suggesting a dose-dependent increase in inflammation in response to α-synuclein.

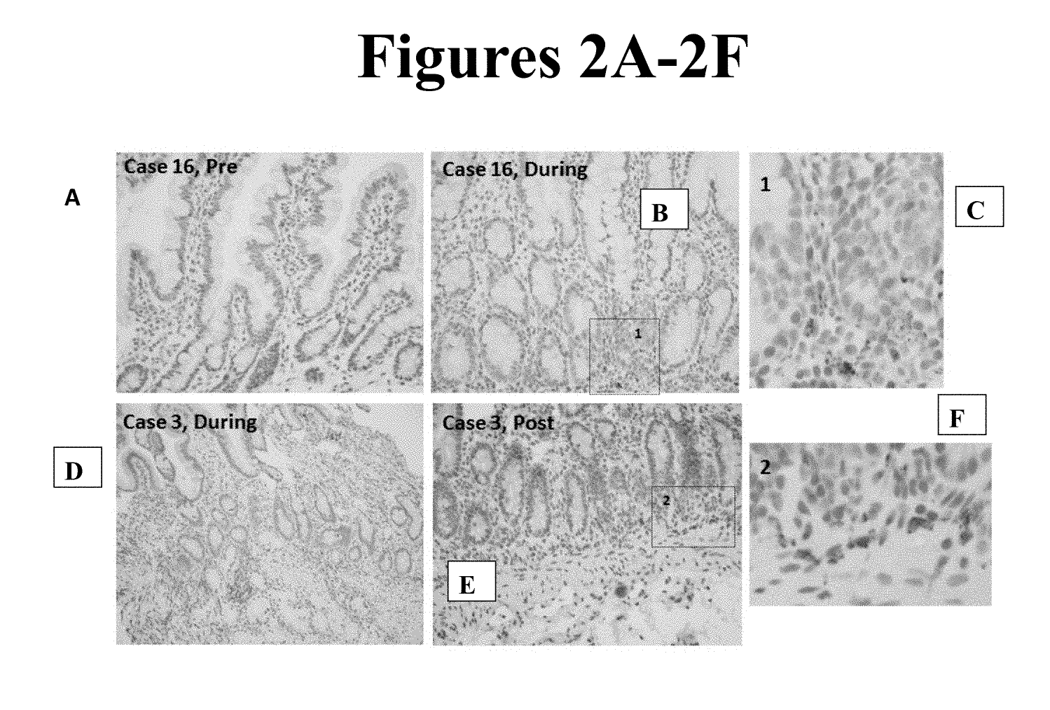

To determine if α-synuclein is induced during viral enteric infection, intestinal biopsies taken as part of the normal standard of care of intestinal allograft recipients were examined. As an immunosuppressed population, these patients are highly susceptible to viral infections. 14 children and 2 adults who received an intestinal transplant and had contracted a Norovirus infection after the surgery were identified. Biopsies were examined that had been taken before, during and after the infection. In most duodenal biopsies sampled during the infection, robust expression of α-synuclein was seen (see e.g., FIG. 2). Many of these patients had documented systemic viral infections, in addition to the documented Noroviral enteritis, which likely accounts for the high level of α-synuclein expression seen in many of the biopsies taken before the Norovirus infection. However, in 4 of the cases, α-synuclein was not seen in tissue from either the native or the transplanted duodenum taken 1 to 6 months prior to the infection, consistent with the hypothesis that the expression of α-synuclein was induced during the Norovirus infection. Tissues taken between 2 weeks to 6 months (mean 2.5 months) following clinical resolution of the infection still exhibited the presence of α-synuclein but generally at lower levels than observed during the period of active infection.

Chemotaxis:

Chemotaxis, or the movement of cells in response to a chemical stimulus, is routinely measured in a Boyden chamber. In this apparatus, immune cells are attached to one side of a membrane containing pores through which the immune cells can traffic. On the other side of the membrane a chemoattractant is introduced. Over the course of several hours the immune cells migrate from the side of the membrane on which they were initially placed to the side exposed to the chemoattractant. Several wells are set up with various concentrations of the chemoattractant. After 24 hours the number of immune cells that have migrated to towards the chemoattractant are counted.

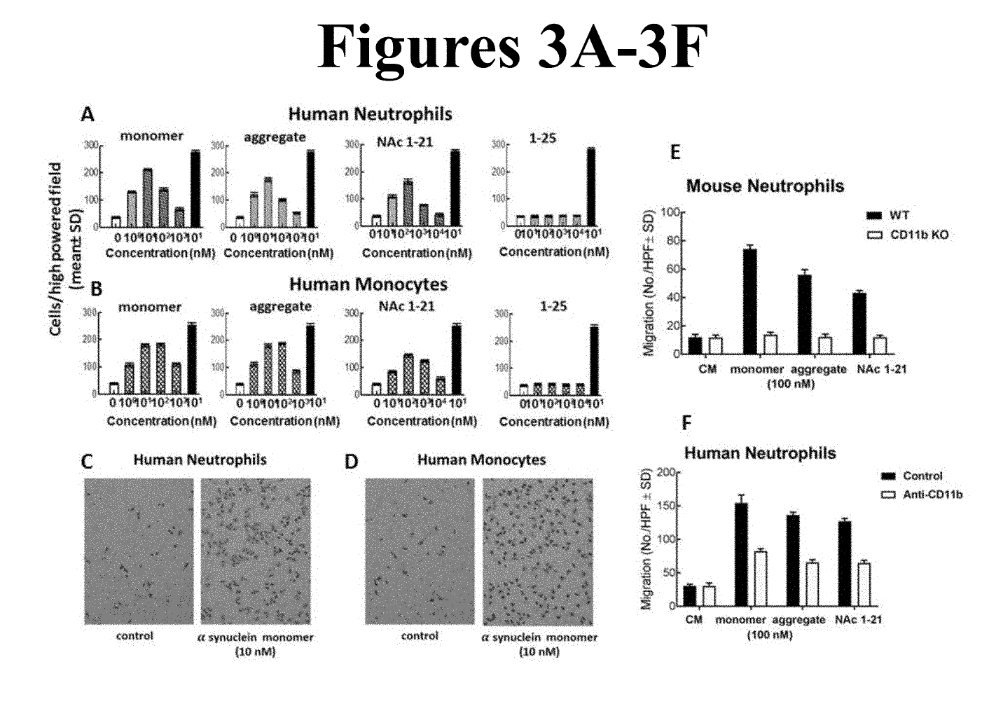

The observation that neutrophils and macrophages colocalized with neurites expressing α-synuclein prompted examination of whether α-synuclein exhibited chemotactic activity. Both monomeric and aggregated recombinant human alpha-synuclein were chemotactic at nanomolar concentrations towards human neutrophils and monocytes exhibiting the classical bell shaped concentration curve characteristic of chemoattractants (see FIGS. 3A-D). Furthermore, the N-acetylated peptide, corresponding to the first 21 amino acids of human alpha-synuclein, which is universally N-acetylated in mammalian cells (Anderson et al., 2006), retained the chemotactic activity of the full-length protein. In contrast, the slightly longer peptide, extending to residue 25 but lacking the N-acetyl moiety, was inactive, implicating N-terminal acetylation as a determinant of the peptide's activity.

It was then explored whether peripheral white blood cells require CD11b to respond to α-synuclein. α-Synuclein is known to be secreted from cultured neurons. While both monomeric and aggregated α-synuclein exhibited robust chemotactic activity towards neutrophils from wild type mice, no chemotactic activity was observed for cells from CD11b deficient mice (FIG. 3E). In a separate experiment, treatment of human neutrophils with an antibody directed at CD11b reduced the chemotactic response to α-synuclein (FIG. 3F).

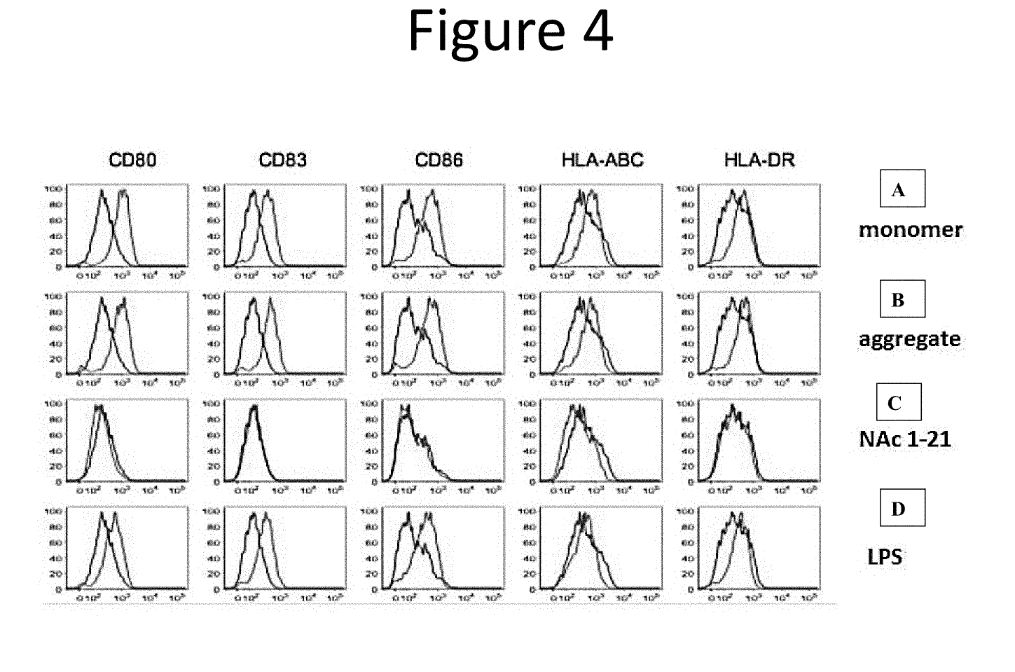

To determine whether alpha-synuclein could activate dendritic cells, human monocyte derived dendritic cells were exposed for 2 days to alpha-synuclein monomer, alpha-synuclein aggregate, and N—Ac 1-21 peptide, and then analyzed by flow cytometry to measure the extent of phenotypic maturation, using CD80, CD83, CD86, HLA-ABC, and HLA-DR as determinants (FIG. 4). While both alpha-synuclein monomer and aggregate promoted dendritic cell maturation, the N-terminal peptide did not, demonstrating that the chemotactic activity of alpha-synuclein and the segment involved in dendritic cell maturation reside on different portions of the alpha-synuclein molecule. Maturation of dendritic cells by the alpha-synuclein monomer was not inhibited by pretreatment of the cells with a blocking antibody directed at TLR4, demonstrating both that the cellular response was independent of the small amount of endotoxin present in the recombinant alpha-synuclein preparation, and that alpha-synuclein does not engage the TLR4 receptor to effect maturation.

The data described herein demonstrates that α-synuclein is present in neurites and cell bodies within the intestinal wall of children with a variety of upper GI infections, including H. pylori gastritis, and esophageal candidiasis. In intestinal transplant recipients, α-synuclein was found in both native and grafted duodenum in all cases during active Norovirus infection. Interestingly, the α-synuclein response of the grafted (vagotomized) tissue was as robust as that of native duodenum. This implies that α-synuclein is produced by the ENS and does not require input from higher centers. Furthermore, it was demonstrated that α-synuclein is both a potent chemoattractant for neutrophils and monocytes and a maturation factor for dendritic cells. Taken together, these findings suggest that α-synuclein is a component of the innate immune response of the human ENS.

Interestingly, the chemotactic potency of α-synuclein oligomers was greater than that of the monomer on a molar basis, which may help explain the increased neurotoxicity associated with α-synuclein aggregates. The N-terminal portion of α-synuclein, comprising the first 21 amino acids, and including the N-terminal acetyl modification, appears to contain the primary chemo-attractive signal. Furthermore, it appears that the integrin subunit CD11b is required for the chemotactic effect of α-synuclein. We showed that monocytes and neutrophils isolated from mice lacking CD11b failed to migrate, or human neutrophils treated with a CD11b blocking antibody exhibit a reduced chemotactic response. CD11b, although an integrin subunit, also binds a diverse array of soluble ligands, including the human antimicrobial peptide, LL-37 which, like α-synuclein, exhibits CD11b dependent chemotactic activity. Indeed, in a recent study, CD11b has been described as an “alarmin receptor” because of its binding affinity for many of the diverse array of peptides and proteins (“alarmins”) that are released by tissues to activate the innate immune system in the setting of injury or infection. Furthermore, through activation of dendritic cells, the nervous system could direct the adaptive immune system to respond to pathogens that have entered its receptive field.

The presence of α-synuclein in non-neural tissues can be understood in a new light. For example, the presence of α-synuclein in erythrocytes, the major source of α-synuclein in blood, suggests that α-synuclein plays a role in vascular injury, likely related to its innate immune functions.

The findings described herein are in contrast to those of Wang et al, who reported that aggregated α-synuclein, while capable of attracting rodent microglia dependent on CD11b, did not exhibit chemotactic activity towards rat monocytes. Wang et al. (2015). In these experiments, aggregated α-synuclein was assayed at a single concentration, 1 μM, which is on the descending dose response of the monocyte.

The discovery that α-synuclein is expressed during infection and provides immune functions leads to the conclusion that induction of this protein within the ENS is part of its normal immune defense mechanism. Indeed, even the transport of α-synuclein, as either monomer or aggregate, from gut to brain appears to occur normally. Human α-synuclein injected into the gastric wall of rodents, for example, is taken up by the vagus and transported to the dorsal motor nucleus within the brainstem in a time-dependent manner. Holmqvist et al. (2014). Since it is known from genetic studies that individuals with multiple copies of α-synuclein invariably develop PD, an increase in the expression of α-synuclein is sufficient to cause PD. Singleton et al. (2003); and Chartier-Harlin et al. (2004). The increase in expression of enteric α-synuclein induced by acute or chronic gastrointestinal infections in childhood could in principle be exacerbated by continued high levels of α-synuclein expression in the presence of infections lasting a sufficiently long period of time. Indeed, epidemiological studies support an association between chronic H. pylori infection and the risk of developing PD. C. J. Barnum, M. G. Tansey (2012); and Nielsen et al. (2012). Strikingly, individuals who have received a full truncal vagotomy (as treatment for peptic ulcer) are at a decreased risk of developing PD. Svensson et al. (2015).

The recent report that individuals with PD have increased intestinal permeability suggests another mechanism that might provoke the expression (or accumulation) of α-synuclein within the ENS (Forsyth et al. (2014)), namely the exposure of the ENS to commensal microbes. Orally administered E. coli producing curli protein, a protein that facilitates bacterial attachment to epithelial cells and subsequent invasion, enhanced α-synuclein deposition in plexi in the gut and in hippocampus and striatum in aged Fischer 344 rats, which spontaneously accumulate α-synuclein within their ENS as they age (Phillips et al. (2009)), as compared to rats exposed to mutant bacteria lacking the capacity to produce curli or to rats exposed to vehicle. Chen et al. (2016). In a study involving α-synuclein overexpressing mice, short chain fatty acids, produced by the intestinal microbiome, increased the presence of α-synuclein aggregates in basal ganglia and substantia nigra and enhanced the motor deficit, as did fecal transplants from patients with P D. Sampson et al. (2016).

The data described herein demonstrates that α-synuclein is a component of the innate immune defensive response of the gut and nervous systems and provides insight into the pathophysiology of certain human chronic inflammatory disorders. With respect to PD, the discovery reported here extends Braak's hypothesis that PD begins in the ENS (Braak et al. (2006)), by proposing that PD results from the excessive response of a normal innate immune component of the nervous system.

### II. Conditions to be Treated

The methods and compositions of the invention can be used to treat or prevent any inflammatory condition caused by excessive expression of alpha synuclein, as measured by elevated concentrations of alpha-synuclein present in tissues or as measured by another marker of inflammation. An “elevated amount” of alpha-synuclein concentration can be determined by comparing the concentration of alpha-synuclein in a patient or subject to be treated to the concentration of alpha-synuclein present in the same tissue type in a healthy subject. Alternatively, a marker of inflammation can be measured either quantitatively or qualitatively to demonstrate effectiveness of the methods of the invention in reducing, minimizing, or eliminating excessive expression of alpha synuclein.

In one embodiment, a difference of at least about 5%, about 10%, about 15%, about 20%, about 25%, about 30%, about 35%, about 40%, about 45%, or about 50% corresponds to an “elevated” amount of expression of neuronal alpha synuclein, as compared to a control (e.g., healthy subject).

Exemplary inflammatory diseases or conditions caused by excessive expression or concentration of alpha synuclein include but are not limited to chronic inflammatory diseases (CID). CIDs are a burden to humans because of life-long debilitating illness, increased mortality and high costs for therapy and care. Other than CIDs, infectious diseases like influenza or scarlet fever typically last only for a short period of time and they normally do not lead to chronic disease sequelae. The main difference between CIDs and acute infectious disease is span of time. While an acute infectious disease or inflammation during wound healing represents an adaptive response to overcome a disease and, thus, to increase life-time reproductive success, a CID is outside the adaptive reaction norm leading to maladaptive responses and a reduction of evolutionary fitness, because the stimulating trigger cannot be removed. Examples of CIDs include, but are not limited to, osteoarthritis, autoimmune diseases, such as lupus and rheumatoid arthritis, allergies, asthma, inflammatory bowel disease and Crohn's disease, systemic lupus erythematosus, atherosclerosis, periodontitis, and ulcerative colitis.

Other examples of diseases and conditions associated with inflammation and caused by excessive expression of alpha synuclein include, but are not limited to, a neurodegenerative disorder (NDD) (e.g., an alpha-synucleinopathy such as Parkinson's disease, Lewy body dementia, multiple system atrophy, amytrophic lateral sclerosis, Huntington's chorea, multiple sclerosis and schizophrenia), asthma, chronic peptic ulcer, tuberculosis, chronic periodontitis, chronic sinusitis, chronic active hepatitis, psoriatic arthritis, gouty arthritis, acne vulgaris, osteoarthritis, autoimmune diseases such as rheumatoid arthritis and lupus, autoinflammatory diseases, systemic lupus erythematosus, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis. Further, uncontrolled inflammation plays a role in almost every major disease, including cancer, heart disease, diabetes, Alzheimer's disease and even depression.

Role of inflammation in disease: When cells are in distress, they release chemicals to alert the immune system. The immune system sends its first responders—inflammatory cells—to trap the offending substance or heal the tissue. As this complex chain of events unfolds, blood vessels leak fluid into the site of the injury, causing the telltale swelling, redness and pain. These symptoms might be uncomfortable, but they are essential for the healing process. However, with chronic inflammation, the body is on high alert all the time. This prolonged state of emergency can cause lasting damage to the heart, brain and other organs. For example, when inflammatory cells are present for an extended period of time in blood vessels, they promote the buildup of dangerous plaque. The body sees this plaque as foreign and sends more of its first responders. As the plaque continues to build, the arteries can thicken, making a heart attack or stroke much more likely. Similarly, inflammation in the brain may play a role in Alzheimer's disease. For many years the brain was thought to be off-limits to inflammation because of the blood-brain barrier a sort of built-in security system—but scientists have proved that immune cells can and do infiltrate the brain during times of distress.

### III. Aminosterols Useful in the Compositions and Methods of the Invention

In some embodiments, the methods of the invention utilize an aminosterol. Exemplary aminosterol compounds and formulations are also described in U.S. Pat. Nos. 8,729,058 and 8,623,416, US 2014-0099281 A1, and US 2014-0328792 A1, all of which are specifically incorporated by reference.

The aminosterol can be selected from the group consisting of: (a) squalamine or a pharmaceutically acceptable salt or derivative thereof; (b) a squalamine isomer; (c) Aminosterol 1436 or a pharmaceutically acceptable salt or derivative thereof; (d) an aminosterol comprising a sterol nucleus and a polyamine, attached at any position on the sterol, such that the molecule exhibits a net charge of at least +1, the charge being contributed by the polyamine; (e) an aminosterol which is a derivative of squalamine modified through medical chemistry to improve biodistribution, ease of administration, metabolic stability, or any combination thereof; (f) an aminosterol modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system; (g) an aminosterol that can inhibit the formation of actin stress fibers in endothelial cells stimulated by a ligand known to induce stress fiber formation, having the chemical structure of Formula I:

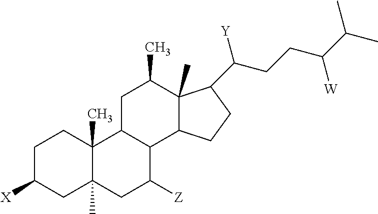

wherein:
W is 24S-OSO₃ or 24R-OSO₃;
X is 3β-H₂N—(CH₂)₄—NH—(CH₂)₃—NH— or 3α-H₂N—(CH₂)₄—NH—(CH₂)₃—NH—;
Y is 20R-CH₃; and
Z is 7α or 7β-OH; or
   (h) any combination thereof. As used herein, the term “aminosterol” is intended to encompass squalamine and derivatives thereof.

The structure of squalamine (C₃₄H₆₅N₃O₅S) is shown below:

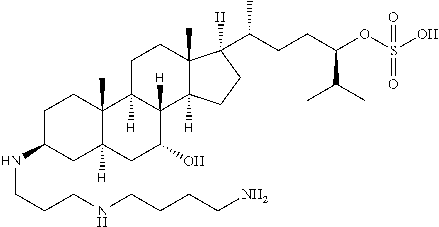

U.S. Pat. No. 6,962,909, for “Treatment of neovascularization disorders with squalamine” to Zasloff et al., discloses various aminosterols, the disclosure of which is specifically incorporated by reference. Any aminosterol known in the art, including those described in U.S. Pat. No. 6,962,909, can be used in the present invention, as long as the aminosterol carries a net positive charge of at least +1 created by a polyamine moiety.

In one embodiment, the methods of the invention can use a formulation of Aminosterol 1436 (Zasloff, Williams et al. 2001) as an insoluble salt of phosphate, polyphosphate, or an organic phosphate ester. In another embodiment, the aminosterol can be composed of a sterol nucleus to which a polyamine is chemically linked, displaying a net positive charge of at least +1. The structure of Aminosterol 1436 is shown below:

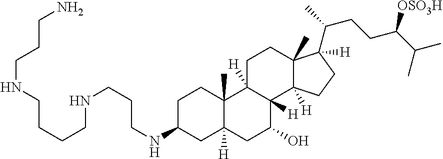

Examples of aminosterols useful in the methods of the invention include squalamine and Aminosterol 1436. A variant or derivative of squalamine, as well as a variant or derivative of Aminosterol 1436, useful in the methods of the invention may have one or more chemical modification which do not modify the therapeutic characteristics of squalamine or Aminosterol 1436. A “variant” or “derivative” of squalamine and/or Aminosterol 1436 is a molecule in which modifications well known in the art of medicinal chemistry to “mimic” the original spatial and charge characteristics of a portion of the original structure have been introduced to improve the therapeutic characteristics of squalamine or Aminosterol 1436, respectively. In general, such modifications are introduced to influence metabolism and biodistribution. Examples of such modifications are given above.

### IV. Dosage Forms

Any pharmaceutically acceptable dosage form may be employed in the methods of the invention. Further, the formulations used in the methods of the invention may conveniently be presented in unit dosage form and may be prepared by any of the methods well known in the art of pharmacy. For example, the composition can be formulated into a dosage form (a) selected from the group consisting of liquid dispersions, gels, aerosols, pulmonary aerosols, nasal sprays, lyophilized formulations, tablets, capsules; and/or (b) into a dosage form selected from the group consisting of controlled release formulations, fast melt formulations, delayed release formulations, extended release formulations, pulsatile release formulations, and mixed immediate release and controlled release formulations; or (c) any combination of (a) and (b).

The methods of the invention can utilize an aminosterol formulation comprising a suspension or a tablet for oral administration. As an oral formulation, squalamine slowly dissolves in the gastrointestinal tract, and does not subject the lining of the intestine to high local concentrations that would otherwise irritate or damage the organ. Similarly, delivery via inhalation or nebularization would provide similar benefits. Fine particles would gradually dissolve in the airway, releasing squalamine into the lungs or nasal passages at non-toxic local concentrations.

An exemplary dosage form is an orally administered dosage form, such as a tablet or capsule. Such methods include the step of bringing into association the aminosterol with the carrier that constitutes one or more accessory ingredients. In general, the formulations are prepared by uniformly and intimately bringing into association the active ingredient with liquid carriers or finely divided solid carriers or both, and then, if necessary, shaping the product. Other examples of exemplary dosage forms include a nasal spray, comprising a dry powder, liquid suspension, liquid emulsion, or other suitable nasal dosage form.

Formulations or compositions of the invention may be packaged together with, or included in a kit with, instructions or a package insert. For instance, such instructions or package inserts may address recommended storage conditions, such as time, temperature and light, taking into account the shelf-life of the aminosterol. Such instructions or package inserts may also address the particular advantages of the aminosterol, such as the ease of storage for formulations that may require use in the field, outside of controlled hospital, clinic or office conditions.

The composition used in the methods of the invention can also be included in nutraceuticals. For instance, an aminosterol composition may be administered in natural products, including milk or milk product obtained from a transgenic mammal which expresses alpha-fetoprotein fusion protein. Such compositions can also include plant or plant products obtained from a transgenic plant which expresses the aminosterol. The aminosterol can also be provided in powder or tablet form, with or without other known additives, carriers, fillers and diluents. Exemplary nutraceuticals are described in Scott Hegenhart, Food Product Design, December 1993.

Any pharmaceutical used for therapeutic administration can be sterile. Sterility is readily accomplished by filtration through sterile filtration membranes (e.g., 0.2 micron membranes).

The invention also provides a pharmaceutical pack or kit comprising one or more containers filled with one or more of the ingredients of the aminosterol composition useful in the methods of the invention, including containers filled with an appropriate amount of a phosphate, either as a powder, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol may be employed in conjunction with other therapeutic compounds.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof. Examples of effervescent agents include effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### V. Dosages

Compositions used in the methods of the invention will be formulated and dosed in a fashion consistent with good medical practice, taking into account the clinical condition of the individual patient (especially the side effects of treatment with the active agent—e.g., aminosterol alone), the method of administration, the scheduling of administration, and other factors known to practitioners. The “effective amount” for purposes herein is thus determined by such considerations.

Effective dosing regimens can be based on the dose required to observe a decrease in α-synuclein amounts following dosing. In one embodiment of the invention, the methods of the invention result in a decrease in the amount of alpha synuclein of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as compared to the amount of α-synuclein present prior to administration of a composition in a method according to the invention.

Effective dosing regimens can in part be established by measuring the rate of excretion of the orally administered aminosterol and correlating this with clinical symptoms and signs. Exemplary dosing regimens include, but are not limited to: (1) Initiating with a “low” initial daily dose, and gradually increasing the daily dose until a dose is reached that results in the desired decrease of inflammation, in vivo α-synuclein amounts, disease or condition adverse effects, or other measureable evidence, where the “low” dose is from about 10 to about 100 mg per person, and the final effective daily dose is between about 25 to about 1000 mg/person; (2) Initiating with a “high” initial dose, which necessarily stimulates the enteric nervous system, and reducing the subsequent daily dosing to that required to elicit a clinically acceptable response, with the “high” daily dose being between about 50 to about 1000 mg/person, and the subsequent lower daily oral dose being between about 25 to about 500 mg/person; (3) Periodic dosing, where an effective dose can be delivered once every about 2, about 3, about 4, about 5, about 6 days, or once weekly, with the initial dose determined to capable of eliciting a clinically acceptable response.

Any therapeutically effective dosage of an aminosterol or a salt or derivative thereof can be used in the methods of the invention. For example, the dosage of an aminosterol or a derivative or salt thereof can be selected from the group consisting of about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150 mg/kg (e.g., dose based upon the weight of the subject to be treated).

In another example, the dosage of an aminosterol or a derivative or salt thereof can be selected from the group consisting of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, or about 110 mg/m² (e.g., dose based upon the body surface area of the subject to be treated).

In another embodiment, the dosage of an aminosterol or a derivative or salt thereof can be selected from the group consisting of about 10 mg to about 400 mg, or about 50 mg to about 350 mg, or about 100 mg to about 300 mg, or about 100 mg to about 200 mg, or any amount in-between these values, such as any amount between 10 and 400 mg, any amount between 50 and 350 mg, any amount between 100 and 300 mg, or any amount between 100 and 200 mg.

Dosing should continue at least until the clinical condition has resolved. To establish the need for continued dosing, treatment can be discontinued and the condition revaluated. If necessary, administration should be resumed. The period of dosing can be for about 1, about 2, about 3, or about 4 weeks; about 1, about 2, about 3, about 4, about 5, about 6, about 7, about 8, about 9, about 10, about 11, or about 12 months, or about 1, about 2, about 3, about 4, or 5 years, or longer.

In other embodiments of the invention, the first or initial “large” dose of aminosterol (per person) can be selected from the group consisting of about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1025, about 1050, about 1075, about 1100, about 1125, about 1150, about 1175, about 1200, about 1225, about 1250, about 1275, about 1300, about 1325, about 1350, about 1375, about 1400, about 1425, about 1450, about 1475, about 1500, about 1525, about 1550, about 1575, about 1600, about 1625, about 1650, about 1675, about 1700, about 1725, about 1750, about 1775, about 1800, about 1825, about 1850, about 1875, about 1900, about 1925, about 1950, about 1975, and about 2000 mg. In other embodiments of the invention, the second smaller dose of aminosterol (per person) is less than the first or initial dose and can be selected from the group consisting of about, 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, and about 1000 mg. Finally, in other embodiments of the invention, the periodic aminosterol dosage (per person) can be selected from the group consisting of about 10, about 25, about 50, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, and about 1000 mg.

Several clinical trials have been conducted relating to the use of squalamine, and which describe exemplary dosages:

(1) ClinicalTrials.gov Identifier NCT01769183 for “Squalamine for the Treatment in Proliferative Diabetic Retinopathy,” by Elman Retina Group, testing the use of 0.2% ophthalmic squalamine lactate solution in the treatment of retinal neovascularization resulting from proliferative diabetic retinopathy, with a total of 6 patients enrolled (study completed) (https://clinicaltrials.gov/ct2/show/NCT01769183?term=squalamine&rank=2);

(2) Clinicaltrials.gov Identifier NCT02727881 for “Efficacy and Safety Study of Squalamine Ophthalmic Solution in Subjects With Neovascular AMD (MAKO),” by Ohr Pharmaceutical Inc., testing the use of 0.2% ophthalmic squalamine lactate solution in the treatment age-related macular degeneration, with 230 subjects enrolled (study is active) (https://clinicaltrials.gov/ct2/show/NCT02727881?term=squalamine&rank=3);

(3) Clinicaltrials.gov Identifier NCT02614937 for “Study of Squalamine Lactate for the Treatment of Macular Edema Related to Retinal Vein Occlusion,” testing the use of 0.2% ophthalmic squalamine lactate solution, by Ohr Pharmaceutical, Inc., with 20 subjects enrolled (study completed) (https://clinicaltrials.gov/ct2/show/NCT02614937?term=squalamine&rank=5);

(4) Clinicaltrials.gov Identifier NCT01678963 for “Efficacy and Safety of Squalamine Lactate Eye Drops in Subjects With Neovascular (Wet) Age-related Macular Degeneration (AMD),” testing the use of 0.2% ophthalmic squalamine lactate solution, by Ohr Pharmaceutical, Inc., with 142 subjects enrolled (study completed) (https://clinicaltrials.gov/ct2/show/NCT01678963?term=squalamine&rank=6);

(5) Clinicaltrials.gov Identifier NCT00333476 for “A Study of MSI-1256F (Squalamine Lactate) To Treat “Wet” Age-Related Macular Degeneration,” evaluating the safety profile of squalamine lactate at doses ranging from 40 mg to 160 mg of squalamine lactate, by Genaera Corporation, with 140 subjects enrolled (study terminated) (https://clinicaltrials.gov/ct2/show/NCT00333476?term=squalamine&rank=7); and

(6) Clinicaltrials.gov Identifier NCT00139282 for “A Safety and Efficacy Study of Squalamine Lactate for Injection (MSI-1256F) for “Wet” Age-Related Macular Degeneration,” by Genaera Corporation, evaluating the safety and efficacy of two doses of Squalamine lactate for Injection administered as intravenous infusions, with an original enrollment of 650 subjects (study terminated) (https://clinicaltrials.gov/ct2/show/NCT00139282?term=squalamine&rank=8).

Several clinical trials have also been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp. (https://clinicaltrials.gov/ct2/show/NCT00509132?term=NCT00509132&rank=1);

(2) ClinicalTrials.gov Identifier NCT00606112 for “A Single Dose, Tolerance and Pharmacokinetic Study in Obese or Overweight Type 2 Diabetic Volunteer,” by Genaera Corp. (https://clinicaltrials.gov/ct2/show/NCT00606112?term=administered+1436&rank=4);

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp. (https://clinicaltrials.gov/ct2/show/NC100806338?term=administered+1436&rank=3); and

(4) ClinicalTrials.gov Identifier: NCT02524951 for “Safety and Tolerability of MSI-1436C in Metastatic Breast Cancer,” by DepyMed Inc. (https://clinicaltrials.gov/ct2/show/NCT02524951?term=NCT02524951&rank=1).

### VI. Combination Therapy

In the methods of the invention, the aminosterol compositions may be administered alone or in combination with other therapeutic agents. As noted above, the methods of the invention are useful in treating and/or preventing the conditions described herein related to inflammatory conditions or diseases. Thus, any active agent known to be useful in treating these conditions can be used in the methods of the invention, and either combined with the aminosterol compositions used in the methods of the invention, or administered separately or sequentially.

Examples of conventional drugs used in treating inflammation-related diseases or conditions include, but are not limited to NSAIDs, steroids, corticosteroids, tocilizumab (Actemra®), certolizumab (Cimzia®), etanercept (Enbrel®), adalimumab (Humira®), anakinra (Kineret®), abatacept (Orencia®), infliximab (Remicade®), and rituximab (Rituxan®). Exemplary corticosteroids include but are not limited to methylprednisolone, prednisone, prednisolone, budesonide, dexamethasone, hydrocortisone, betamethasone, cortisone, prednisolone, and triamcinolone. In such a combination therapy, conventional drug can be administered before or after the composition according to the invention, or the conventional drug can be combined with the composition of the invention for simultaneous administration.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

### VII. Definitions

The following definitions are provided to facilitate understanding of certain terms used throughout this specification.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### VIII. Examples

### Example 1

To determine if α-synuclein expression in the enteric nervous system (ENS) is associated with infection and therefore inflammation, biopsies taken over a 9-year period from children evaluated for clinically significant upper GI distress warranting endoscopy were examined. A pediatric population was selected because it would avoid the bias of an adult population with “pre-clinical Parkinson's disease (PD).”

Biopsies were obtained as part of standard clinical practice. Protocols were approved by the Institutional Review Board at each participating center. Endoscopic biopsy specimens were retrieved from 42 children (mean age 12.4 years) with upper GI distress with the pathological diagnoses of duodenitis, gastritis, H. pylori, and reactive gastropathy at one academic center over a 9-year period. A second population included endoscopic biopsies obtained from 14 pediatric (mean age 3.4 years) and 2 adult intestinal transplant recipients (mean age 40 years) who developed a documented Norovirus infection (N=16). In this immunosuppressed population, viral infections occur frequently, and biopsies of the allograft are routinely obtained prospectively at specific times following transplantation and when clinically indicated.

Inflammation:

Most of the 42 pediatric cases exhibited pathological evidence of acute and/or chronic mucosal inflammation, as determined by the presence of neutrophil or mononuclear cell infiltrates, respectively. An infectious organism could be implicated as the cause of an inflammatory response in 23/42 (55%) and included H. pylori in 20/23 (87%) and Candida in 2/23 (9%).

Immunohistochemistry and Scoring:

Tissue preparation followed published procedures. Fishbein et al. (2008). Serial sections were cut at 4 micron thickness. The primary antibodies used were: alpha-synuclein (LB 509, Abcam ab27766), PGP9.5 (Agilent Z5116), and CD68 (Abcam ab955). The secondary antibody was Envision Plus Polymer anti-mouse conjugated to HRP (Agilent K4001). HRP was visualized using DAB chromagen (Agilent K3468) following the manufacturer's protocol.

Histological scoring was performed on two sections per block for each stain by two pathologists blinded to the diagnoses. Mucosa from each biopsy was assessed by H&E stain for acute (polymorphonuclear) and chronic (mononuclear) inflammation (0=no inflammatory change; 1=minimal inflammatory change; 2=moderate inflammatory change; 3=marked inflammatory change), and for PGP 9.5—positive neurite density (1=minimal; 2=low; 3=moderate; 4=high), neurite α-synuclein presence and intensity (0=no staining; 1=slight; 2=low; 3=moderate; 4=high), and CD68 positive cell number (1=rare cells; 2=low cell number; 3=moderate cell number; 4=high cell number). The scoring of each pathologist was averaged. Results were expressed as mean+/−standard deviation and analyzed using Student t-test.

Chemotaxis and Dendritic Cell Maturation:

Chemotaxis assays followed published procedures. De et al. (2000). Neutrophils and monocytes were isolated from the blood of healthy human donors as described. Id. Recombinant human α-synuclein (lot #20162050, endotoxin contamination 18.6 EU/mg) was from Proteos, Inc. via the Michael J. Fox Foundation. The α-synuclein aggregates were made following a protocol provided by Proteos. N-terminal peptides were synthesized by solid phase chemistry and purified to >98% purity by high performance liquid chromatography. Male CD11b−/− (B6.12954-Itgamtm1Myd/J) and wild type C57B6 control mice were from The Jackson Laboratory (Bar Harbor, Me.). Anti-human CD11b antibody (clone M1/70, ultrapure rat IgG) was from BioLegend (San Diego, Calif.). Dendritic cell maturation studies were conducted as reported. Yang et al. (2012).

The biopsies were immunostained for α-synuclein. Since the extent of α-synuclein staining depends both on its intraneuronal concentration as well as on numbers of neurites present within the tissue specimen, serial sections were also immunostained for the neural protein PGP 9.5. In several specimens, the neuronal localization of α-synuclein by immunofluorescent co-localization of α-synuclein and PGP 9.5 was confirmed. Because many of the gastric biopsies had insufficient neural tissue to evaluate, the analyses were focused on duodenal specimens.

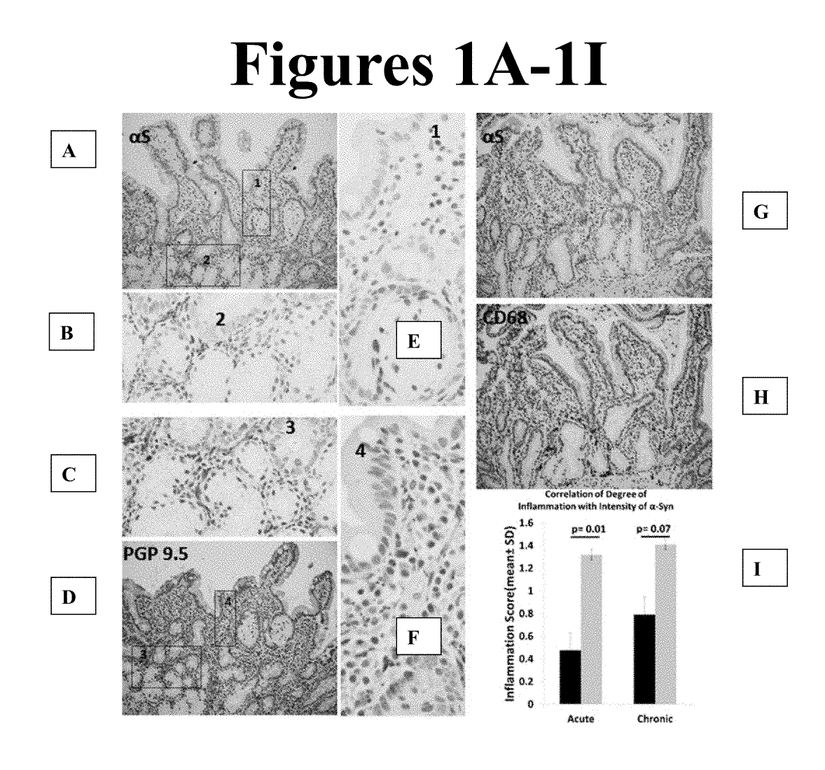

α-Synuclein was expressed in 19/23 (83%) of duodenal biopsies with a confirmed bacterial or fungal infection (FIG. 1). By contrast, in a recent report, α-synuclein staining in the ENS of the gastric mucosa of subjects over the age of 70 was noted in about 60% of individuals with PD, but in fewer than 10% of an aged matched control population. In all tissue specimens examined, neuronal processes that expressed α-synuclein could be seen adjacent to neighboring neuronal processes that did not (FIG. 1). Generally, the distribution of macrophages within the lamina propria overlapped with the areas in which α-synuclein was expressed (FIG. 1) and the inflammatory cell density tended to be proportional to the intensity and extent of nearby α-synuclein staining, suggesting a dose-dependent increase in inflammation in response to α-synuclein (FIG. 1I).

To determine if α-synuclein is induced during viral enteric infection, intestinal biopsies taken as part of the normal standard of care of intestinal allograft recipients were examined. As an immunosuppressed population, intestinal allograft recipients are highly susceptible to viral infections. 14 children and 2 adults who received an intestinal transplant and had contracted a Norovirus infection after the surgery were identified, definitively diagnosed by PCR. Biopsies were examined that had been taken before, during and after the infection. In most duodenal biopsies sampled during the infection robust expression of α-synuclein was seen (FIG. 2). Many of these patients had documented systemic viral infections, in addition to the documented Noroviral enteritis, which likely accounts for the high level of α-synuclein expression seen in many of the biopsies taken before the Norovirus infection. However, in 4 of the cases, α-synuclein was not seen in tissue from either the native or the transplanted duodenum taken 1 to 6 months prior to the infection, consistent with the hypothesis that the expression of α-synuclein was induced during the Norovirus infection (FIG. 2). Tissues taken between 2 weeks to 6 months (mean 2.5 months) following clinical resolution of the infection still exhibited the presence of α-synuclein but generally at lower levels than observed during the period of active infection (FIG. 2).

The observation that neutrophils and macrophages colocalized with neurites expressing α-synuclein prompted the examination of whether α-synuclein exhibited chemotactic activity. Indeed, both monomeric and aggregated recombinant human α-synuclein were chemotactic towards human neutrophils and monocytes exhibiting the classical bell shaped concentration curve characteristic of chemoattractants (FIGS. 3A-3D). Furthermore, the N-acetylated peptide, corresponding to the first 21 amino acids of human α-synuclein, which is universally N-acetylated in mammalian cells, retained the chemotactic activity of the full-length protein. In contrast, the slightly longer peptide, extending to residue 25 but lacking the N-acetyl moiety was inactive, implicating N-terminal acetylation as a determinant of the peptide's activity.

### Example 2

The purpose of this example was to determine whether α-synuclein could activate dendritic cells.

Human monocyte derived dendritic cells were exposed for 2 days to α-synuclein monomer, aggregate, and N—Ac 1-21 peptide and then analyzed by flow cytometry to measure the extent of phenotypic maturation, using CD80, CD83, CD86, HLA-ABC, and HLA-DR as determinants (FIG. 4).

While both α-synuclein monomer and α-synuclein aggregate promoted dendritic cell maturation, the N-terminal peptide did not, demonstrating that the chemotactic activity of α-synuclein and the segment involved in dendritic cell maturation reside on different portions of the molecule. Maturation of dendritic cells by the monomer was not inhibited by pretreatment of the cells with a blocking antibody directed at TLR4, demonstrating both that the cellular response was independent of the small amount of endotoxin present in the recombinant α-synuclein preparation, and that α-synuclein does not engage the TLR4 receptor to effect maturation.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents. Throughout the specification, any and all references to a publicly available document, including a U.S. patent, are specifically incorporated by reference.

### REFERENCES

1. Anderson et al., “Phosphorylation of Ser-129 is the dominant pathological modification of alpha-synuclein in familial and sporadic Lewy body disease,” J. Biol. Chem., 281: 29739-29752 (2006).
2. Barbour et al., “Red blood cells are the major source of alpha-synuclein in blood,” Neurodegener. Dis., 5: 55-59 (2008).
3. C. J. Barnum, M. G. Tansey, “Neuroinflammation and non-motor symptoms: the dark passenger of Parkinson's disease?” Curr. Neurol. Neurosci. Rep., 12: 350-358 (2012).
4. Beatman et al., “Alpha-Synuclein Expression Restricts RNA Viral Infections in the Brain,” J. Virol., 90: 2767-2782 (2015).
5. Braak, et al., “Gastric alpha-synuclein immunoreactive inclusions in Meissner's and Auerbach's plexuses in cases staged for Parkinson's disease-related brain pathology,” Neuroscience letters, 396: 67-72 (2006).
6. Burre, J., “The Synaptic Function of alpha-Synuclein,” J. of Parkinson's Dis., 5: 699-713 (2015).
7. Chartier-Harlin et al., “Alpha-synuclein locus duplication as a cause of familial Parkinson's disease,” Lancet, 364: 1167-1169 (2004).
8. Chen et al., “Exposure to the Functional Bacterial Amyloid Protein Curli Enhances Alpha-Synuclein Aggregation in Aged Fischer 344 Rats and Caenorhabditis elegans,” Sci. Rep., 6: 34477 (2016).
9. De et al., LL-37, “The neutrophil granule- and epithelial cell-derived cathelicidin, utilizes formyl peptide receptor-like 1 (FPRL1) as a receptor to chemoattract human peripheral blood neutrophils, monocytes, and T cells,” J. Exp. Med., 192: 1069-1074 (2000).
10. K. Del Tredici, H. Braak, “Review: Sporadic Parkinson's disease: development and distribution of alpha-synuclein pathology,” Neuropathol. Appl. Neurobiol., 42: 33-50 (2016).
11. Fishbein et al., “NOD2-expressing bone marrow-derived cells appear to regulate epithelial innate immunity of the transplanted human small intestine,” Gut, 57, 323-330 (2008).
12. Forsyth et al., “Increased intestinal permeability correlates with sigmoid mucosa alpha-synuclein staining and endotoxin exposure markers in early Parkinson's disease,” PLoS One, 6: e28032 (2011).
13. Fujiwara et al., “alpha-Synuclein is phosphorylated in synucleinopathy lesions,” Nature Cell Biol., 4 (2): 160-4 (2002).
14. Holmqvist et al., “Direct evidence of Parkinson pathology spread from the gastrointestinal tract to the brain in rats,” Acta. Neuropathol., 128: 805-820 (2014).
15. Kabaria et al., “Inhibition of miR-34b and miR-34c enhances α-synuclein expression in Parkinson's disease,” FEBS Lett., 589(3):319-25 (2015).
16. Kelly et al., “Progression of intestinal permeability changes and alpha-synuclein expression in a mouse model of Parkinson's disease,” Movement disorders: official J. of the Movement Disorder Soc., 29: 999-1009 (2014).
17. Kisos et al., “Increased neuronal alpha-synuclein pathology associates with its accumulation in oligodendrocytes in mice modeling alpha-synucleinopathies,” PLoS One, 7: e46817 (2012).
18. McGeer et al., “Reactive microglia are positive for HLA-DR in the substantia nigra of Parkinson's and Alzheimer's disease brains,” Neurology, 38:1285-1291 (1988).
19. McGeer et al., “Inflammation, the complement system and the diseases of aging,” Neurobiol. Aging, 26(Suppl 1): 94-97 (2005).
20. Nielsen et al., “Treatment for Helicobacter pylori infection and risk of Parkinson's disease in Denmark,” Eur. J. Neurol., 19: 864-869 (2012).
21. Phillips, et al., “Alpha-synuclein immunopositive aggregates in the myenteric plexus of the aging Fischer 344 rat,” Exp. Neurol., 220: 109-119 (2009).
22. Podolnikova et al., “Ligand recognition specificity of leukocyte integrin alphaMbeta2 (Mac-1, CD11b/CD18) and its functional consequences,” Biochemistry, 54:1408-1420 (2015).
23. Rao et al., “Aminosterols from the dogfish shark Squalus acanthias,”J. Nat. Prod., 63(5): 631-5 (2000).
24. Sampson et al., “Gut Microbiota Regulate Motor Deficits and Neuroinflammation in a Model of Parkinson's Disease, Cell, 167: 1469-1480 e1412 (2016).
25. Sanchez-Ferro et al., “In vivo gastric detection of alpha-synuclein inclusions in Parkinson's disease,” Movement disorders: Official J. of the Movement Disorder Soc., 30: 517-524 (2015).
26. Shinnar et al., “Squalamine Family of Aminosterol Antibiotics from Various Shark Species,” The FASEB 1, 21:791.4 (2007).
27. Singleton et al., “alpha-Synuclein locus triplication causes Parkinson's disease,” Science, 302: 841 (2003).
28. Svensson et al., “Does vagotomy reduce the risk of Parkinson's disease: The authors reply,” Ann. Neurol., 78: 1012-1013 (2015).
29. Timpone et al., “Resistant cytomegalovirus in intestinal and multivisceral transplant recipients,” Transpl. Infect. Dis., 18: 202-209 (2016).
30. van Rooijen et al., “Tryptophan fluorescence reveals structural features of alpha-synuclein oligomers,” J. of Mol. Biol., 394 (5): 826-33 (2009).
31. Wang et al., “alpha-Synuclein, a chemoattractant, directs microglial migration via H2O2-dependent Lyn phosphorylation,” PNAS, USA, 112: E1926-1935 (2015).
32. Yang et al., “High-mobility group nucleosome-binding protein 1 acts as an alarmin and is critical for lipopolysaccharide-induced immune responses,” J. Exp. Med., 209: 157-171 (2012).
33. Ueda et al., “Molecular cloning of cDNA encoding an unrecognized component of amyloid in Alzheimer disease,” PNAS, USA, 90(23): 11282-6 (1993).
34. Zasloff et al., “A spermine-coupled cholesterol metabolite from the shark with potent appetite suppressant and antidiabetic properties,” Int. J. Obes. Relat. Metab. Disord., 25(5): 689-97 (2001).
35. Zhang et al., “Aggregated alpha-synuclein activates microglia: a process leading to disease progression in Parkinson's disease,” FASEB J., 19: 533-542 (2005).

What is claimed:

1. A method of treating or preventing an inflammatory disease or condition caused by excessive expression or concentration of alpha synuclein in a subject, comprising administering to the subject a composition that results in reducing the concentration of alpha synuclein in tissue or at a site of inflammation.

2. The method of claim 1, wherein the tissue is from a site of inflammation.

3. The method of claim 1, wherein the tissue is selected from the group consisting of gastrointestinal (GI) tract, skin, lungs, liver, kidney, heart, or joint synovial membranes.

4. The method of claim 1, wherein the decrease in alpha-synuclein concentration in is measured qualitatively, quantitatively, or semi-quantitatively by one or more methods selected from the group consisting of:
(a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control;
(b) measuring the intensity of inflammation over time;
(c) measuring the amount of inflammatory markers over time;
(d) measuring the amount of inflammatory markers in blood, plasma, or tissue over time, either qualitatively or quantitatively;
(e) measuring the amount of one or more inflammatory marker cytokines in blood, plasma, or tissue over time, either qualitatively or quantitatively;
(f) measuring the amount of one or more plasma markers of inflammation such as TNF, IL-8, or CRP in blood, plasma, or tissue over time, either qualitatively or quantitatively; and
(g) measuring the amount of inflammatory cells in blood, plasma, or tissue over time, either qualitatively or quantitatively.

5. The method of claim 1, wherein the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment.

6. The method of claim 5, wherein the decrease is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

7. The method of claim 1, wherein the composition that reduces the concentration of alpha synuclein comprises an effective amount of an aminosterol, miR-34b, or miR-34c.

8. The method of claim 7, wherein the aminosterol:
(a) is a natural aminosterol isolated from the liver of Squalus acanthias;
(b) is a squalamine isomer;
(c) comprises a sterol nucleus and a polyamine, attached at any position on the sterol, such that the molecule exhibits a net charge of at least +1, the charge being contributed by the polyamine;
(d) comprises a bile acid nucleus and a polyamine, attached at any position on the bile acid, such that the molecule exhibits a net charge of at least +1, the charge being contributed by the polyamine;
(e) is modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; or
(f) is a derivative of squalamine or natural aminosterol modified through medical chemistry to improve bio-distribution, ease of administration, metabolic stability, or any combination thereof.

9. The method of claim 7, wherein the aminosterol is squalamine or Aminosterol 1436.

10. The method of claim 7, wherein the effective amount of the aminosterol is selected from the group consisting of:
(a) about 0.1 to about 20 mg/kg body weight;
(b) about 0.1 to about 150 mg/kg body weight;
(c) about 10 to about 100 mg/subject;
(d) about 10 mg to about 400 mg/subject;
(e) about 25 to about 1000 mg/subject;
(f) about 25 to about 500 mg/subject;
(g) about 50 to about 350 mg/subject; and
(h) about 1 to about 110 mg/m².

11. The method of claim 7, wherein the aminosterol is administered in combination with at least one additional active agent to achieve either an additive or synergistic effect.

12. The method of claim 11, wherein the additional active agent is administered via a method selected from the group consisting of
(a) concomitantly;
(b) as an admixture;
(c) separately and simultaneously or concurrently; and
(d) separately and sequentially.

13. The method of claim 7, wherein the aminosterol composition further comprises at least one pharmaceutically acceptable carrier.

14. The method of claim 1, wherein the subject is human.

15. The method of claim 1, wherein the method is applied to a patient population susceptible to excessive expression of alpha-synuclein, resulting in an excessive or high concentration of alpha-synuclein.

16. A method of treating or preventing an inflammatory disease or condition caused by excessive expression or concentration of alpha synuclein, comprising administering to a subject in need an active agent that binds with alpha-synuclein to form a physical complex.

17. The method of claim 16, wherein binding of alpha-synuclein to the active agent prevents the interaction of alpha-synuclein with CD11b.

18. The method of claim 16, wherein the active agent is an antibody that specifically binds to alpha-synuclein.

19. The method of claim 16, wherein the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment.

20. A method of treating or preventing an inflammatory disease or condition caused by excessive expression or concentration of alpha synuclein, comprising administering to a subject in need an active agent that binds to CD11b to form a physical complex.

21. The method of claim 20, wherein binding of the active agent to CD11b prevents the interaction of alpha-synuclein with CD11b.

22. The method of claim 20, wherein the active agent is an antibody that specifically binds to CD11b.

23. The method of claim 20, wherein the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment.

24. A method of identifying a subject with a condition amenable to treatment targeting alpha-synuclein CD11b interaction, comprising:
(a) obtaining a tissue sample from a site of inflammation from the subject; and
(b) qualitatively, quantitatively or semi-quantitatively determining the concentration of alpha-synuclein within the tissue sample;
wherein an elevated concentration of alpha-synuclein present in the tissue as compared to a control, indicates that the subject is amenable to treatment targeting alpha-synuclein CD11b interaction.
